(19) United States
(12) Patent Application Publication
(10) Pub. No.: US 2006/0201520 A1
(43) Pub. Date: Sep. 14, 2006

(54) Multifunctional mouthpiece system

(76) Inventor: Robert W. Christensen III, Arvada, CO (US)

Correspondence Address: HOLME ROBERTS & OWEN, LLP, 299 SOUTH MAIN, SUITE 1800, SALT LAKE CITY, UT, 84111, US

(21) Appl. No.: 11/373,345

(22) Filed: Mar. 10, 2006

Related U.S. Application Data
(60) Provisional application No. 60/660,684, filed on Mar. 10, 2005.

(51) Int. Cl. A61F 5/56; A61C 5/14

(52) U.S. Cl. 128/848; 128/860; 128/861

(57) ABSTRACT

A mouthpiece system includes a bite area shaped to fit a general curved pattern of a user's dentition. The bite area has an occlusal area arranged to contact the user's dentition when inserted into a user's mouth. The occlusal area includes a palatal edge on a side proximate the user's throat, a labial edge on another side proximate the user's lips, and a molar edge proximate the user's molar teeth. The system also includes an interface area having a raised body. An extension bar is connected between the bite area and the interface area. A method includes forming the bite area, the interface area, and the extension bar between the bite area and the interface area.

### CROSS-REFERENCE TO RELATED APPLICATIONS

The present application claims priority to U.S. Provisional Patent Application No. 60/660,684, filed Mar. 10, 2005, entitled “Multifunctional Mouthpiece” which application is incorporated herein by reference in its entirety.

### BACKGROUND OF THE INVENTION

1. Field of Invention

This invention generally relates to treatment of the jaw, teeth and face. More specifically, the invention relates to a multifunctional mouthpiece.

2. Discussion of Related Art

Dentists use various mouthpieces, splints, and trays in their practice. Mouthpieces generally include forms constructed by dentists to fit a patient's mouth. These mouthpiece forms can be roughly divided into three general types: custom-splints, trays, and mouthguards. Custom-splints are individually made to conform to the user's teeth from an impression mold and are used as retainers and protective mouthpieces. Trays are used to take impressions for molds, teeth bleaching, and other dental procedures. Mouthguards are often used to protect the teeth from impacts, grinding and clenching.

Custom-made splints are generally made of hard plastic, and often acrylic. They are formed to the shape of a patient's teeth in a dental lab from a dental mold. Custom made splints can be made to tightly grip the teeth. However, custom splints are expensive and laborious to produce. An inaccurate mold can lead to a misfit, which requires specialized skill to fit, adjust and/or replace the splint. These splints have thick bases, which are usually too large for patients with a limited mouth opening. Custom made splints can also move teeth, which can lead to other dental or joint problems. If made just from a mold, splints do not contain a structure to adapt to a separate device without adding other parts and specialized products, like those used with the jaw continuous passive motion device of U.S. Pat. No. 4,883,046.

In contrast to custom splints, trays are used to hold a molding material for bleaching or to take an impression of a user's teeth. For insertion and removal by the clinician or user, trays usually include a flat bar that extends from the mouthpiece out of the mouth. Impression trays come in various sizes and shapes to fit different mouth sizes and oral areas. Some trays have small holes throughout their surface for improved impression results. Trays have tall rims along their edges and a wide base, which make them uncomfortable and too big for users with a limited range of jaw motion. The trays also lack structure for adapting an independent device to them.

Mouthguards are primarily made of soft plastic. Some are heat moldable to improve retention in the mouth. Mouthguards are used to minimize dental damage from collisions to the head and mouth, like those used in sports. Mouthguards are designed to be thick to mitigate serious damage and trauma. Some mouthpieces fit over the teeth with no member extending outside the mouth. Others included a pliable tether that extends from the teeth out of the mouth and loops around a helmet. Conventional mouthguards lack the rigidity needed to manipulate the mouth and jaw. They can be uncomfortable, and/or jab into a user's gums and lips.

Mouthpieces in previous devices for exercising, stretching and generally manipulating the jaw were custom designed to include a splint, clutch or mouthpiece for the subject. These devices included a jaw continuous passive motion device as discussed in U.S. Pat. No. 4,883,046, the press-spreader as discussed in U.S. Pat. No. 5,035,420 and the neumatic-spreader as discussed in U.S. Pat. No. 5,562,105. These conventional mouthpieces often require various sizes to fit the variety of different users. They can require extensive customization or extra components for retention. These mouthpieces occlude against the front teeth and they are often too thick for patients with severely limited openings.

### SUMMARY OF THE INVENTION

In accordance with the present invention, a mouthpiece system includes a bite area shaped to fit a general curved pattern of a user's dentition. The bite area has an occlusal area arranged to contact the user's dentition when inserted into a user's mouth. The occlusal area includes a palatal edge on a side proximate the user's throat, a labial edge on another side proximate the user's lips, and a molar edge proximate the user's molar teeth. The system also includes an interface area having a raised body. An extension bar is connected between the bite area and the interface area.

A method of making a mouthpiece system includes forming a bite area shaped to fit a general curved pattern of a user's dentition. The bite area includes an occlusal area arranged to contact the user's dentition when inserted into a user's mouth. The occlusal area has a palatal edge on a side proximate the user's throat, a labial edge on another side proximate the user's lips, and a molar edge proximate the user's molar teeth. The method also includes forming an interface area including a raised body. An extension bar is formed between the bite area and the interface area.

These and other features of the present invention will become more fully apparent from the following description and appended claims, or may be learned by the practice of the invention as set forth hereinafter.

### BRIEF DESCRIPTION OF DRAWINGS

In order to more fully understand the manner in which the above-recited and other advantages and objects of the invention are obtained, a more particular description of the invention will be rendered by reference to specific embodiments thereof which are illustrated in the appended drawings. Understanding that these drawings depict only typical embodiments of the invention and are not therefore to be considered as limiting of its scope, the invention will be described and explained with additional specificity and detail through the use of accompanying drawings.

FIG. 1 is a perspective drawing of a mouthpiece system in accordance with an embodiment of the present invention.

FIG. 2 is a perspective drawing of the mouthpiece system in accordance with another embodiment of the present invention.

FIG. 3 is a perspective drawing of a further embodiment of the present invention showing upper and lower mouthpiece portions placed against each other.

FIG. 4 is a detailed perspective view of a further embodiment of the mouthpiece system having a coating.

FIG. 5 is a detailed perspective view of a further embodiment of the mouthpiece system having retention members.

FIG. 6 is a detailed perspective view of a further embodiment of the mouthpiece system having detachable retention members.

FIG. 7 is a detailed perspective view of a further embodiment of the mouthpiece system having an extended oral cavity.

FIGS. 8-11 are side perspective views of further embodiments of the mouthpiece system having various arrangements of the interface area.

FIG. 12 is a perspective view of a further embodiment of the mouthpiece system having various arrangements of the interface area.

FIG. 13 is a perspective view of a further embodiment of the mouthpiece system having various arrangements of the interface area and a handle.

FIG. 14 is a detailed perspective view of a further embodiment of the mouthpiece system having an extension bar with ribs.

FIGS. 15-16 are perspective views of further embodiments of the mouthpiece system having a detachable structure.

FIG. 17 is a perspective view of a further embodiment of the mouthpiece system having upper and lower mouthpiece portions connected to a push-pull cable system.

### DRAWING REFERENCE NUMERALS

1 mouthpiece system

2 upper mouthpiece portion

4 lower mouthpiece portion

6 bite area

8 labial edge

10 palatal edge

12 molar edge

14 occlusal area

16 tongue cavity

18 extended oral cavity

20 retention member

22 labial rail

22′ modified labial rail

24 palatal rail

26 pad

28 extension bar

30 buttress

32 split

34 flared edge

36 resting surface

38 interface area

40 raised body

42 flat side

43 pivots

44 convex surface

45 slots

46 side surface

48 spherical body

48′ spherical body

50 cavity

52 widened surface

54 sloping surface

56 parallelepiped shape

58 rectangular cross-section

60 geometric configuration

62 groove

64 threads

66 partial bore

68 dimple inverted

68′ dimple raised

70 nubs

72 coating

74 sockets

76 pins

78 notch

80 handle

80′ threaded extension

82 open finger loop

84 closed finger loop

86 ridges

88 insert

90 clip structure

92 fastener

94 bore

96 receiver

98 clasp plug

100 clasp socket

102 push-pull cable system

104 cable

106 threaded cable cap

108 cavity

110 conduit

112 threaded conduit cap

114 cavity

116 drive and control system

### DETAILED DESCRIPTION OF PREFERRED EMBODIMENTS

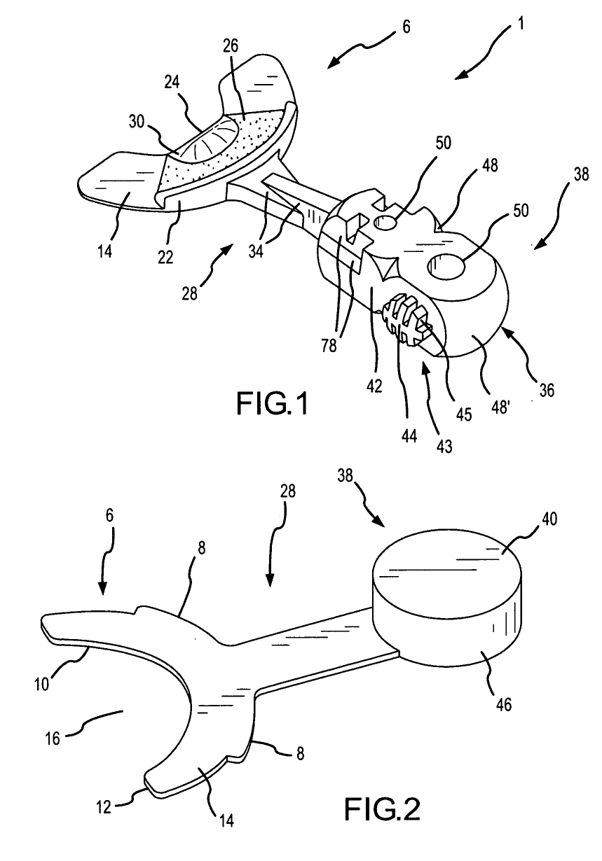

The present invention relates to a multifunctional mouthpiece system and method. The various embodiments of the present invention will now be discussed with respect to the accompanying drawings. In FIGS. 1 and 2, embodiments of the mouthpiece system 1 are illustrated with a bite area 6, which is an area shaped to fit the general curved pattern of a user's mouth, oral opening, occlusion or dentition. The general curved pattern is the general shape of a curve taken from one side of a person's mouth, oral opening, occlusion, or dentition to the other side. The curved pattern can include a partial distance along one side or the other, or can be a symmetric curve from the front, center point of the person's mouth to a distance back toward the person's molars. The bite area 6 is connected to an interface area 38 by an extension bar 28. The extension bar 28 is an elongate member that provides rigid support between the bite area 6 and the interface body 40. The bite area 6 is attached to one end of the extension bar 28 and the interface body 40 is attached to an opposing end of the extension bar 28.

The bite area 6 is generally shaped to fit the user's mouth as discussed above and includes portions defining a labial edge 8, a palatal edge 10, a molar edge 12, an occlusal area 14, and a tongue cavity 16. From the center, the bite area 6 curves in both directions along the user's teeth and around the tongue cavity 16. The mouthpiece system 1 is formed to rest against the dentition of a user at the bite area 6, which is shaped to accommodate various dental structures and instruments. The labial edge 8 is shaped to fit the lips of the user, which is basically a curved-shaped. The palatal edge 10 is arranged to fit the palate, throat and inside of the user's mouth. The molar edge 12 is arranged to sit along and toward the molar or back teeth of the user. The occlusal area 14 is shaped to follow the curvature of the user's teeth.

At the opposing end of the mouthpiece system 1 is the interface area 38. The interface area 38 includes a raised body 40 with a predetermined shape. The extension bar 28 connects the bite area 6 at the labial edge 8 to the interface area 38. The extension bar 28 can include flared edges 34 where it meets the bite and interface areas 6, 38 to provide extra support. As shown in FIG. 1, the extension bar 28 can be unadorned and continuous, flaring or widening from the middle area horizontally to the bite area 6 and vertically to the interface area 38.

The embodiment shown in FIG. 2 has a flat bite area 6. By providing mouthpiece system 1 with a flat bite area 6, the system can be used in tight applications where raised components would otherwise inhibit its use. The mouthpiece system 1 can be used independently or integrated with another device for interaction with a user's mouth and jaw.

In the present invention, the mouthpiece system 1 can be made of an ABS plastic, a non-toxic durable plastic like polypropylene or urethane, or other types of materials known by one skilled in the art that provides moldable, rigid or semi-rigid properties suitable for insertion into a user's mouth. For example, the mouthpiece system 1 can also be made of rubber, various natural materials, or various plasticized materials.

Another embodiment of the mouthpiece system 1 is shown in FIG. 1. The bite area 6 is shaped with a predetermined formation for insertion under the user's teeth. The bite area 6 includes an occlusal area 14 that is substantially flat having a thickness between about 0.01 mm to about 5.0 mm. The bite area 6 also can include retention members 20 to retain the user's teeth. The retention members 20 can include a labial rail 22 and a palatal rail 24. The labial rail 22 extends out from the occlusal area 14 to form a wall-like structure that conforms to the labial edge 8. The labial rail 22 rises above the occlusal area 14 about 0.01 mm to about 10.0 mm. In the exemplary embodiment of the present invention, the labial rail 22 is about 5.5 mm tall, from edge to edge.

The palatal rail 24 is shaped to conform to the palatal edge 10. The palatal rail 24 can have a wall-like structure that slopes down onto the occlusal area 14. The height of the palatal rail 24 from edge to edge can be about 0.01 mm to about 5.0 mm. In this exemplary embodiment, the palatal rail 24 is about 2.0 mm high. If the occlusal area 14 extends uncomfortably into the sides of the user's mouth or is too large to fit within a user's mouth it can be trimmed. The mouthpiece system 1 in the exemplary embodiment is made with a thin occlusal area 14 to allow a user to easily trim it if necessary. The occlusal area 14 in this embodiment is about 1.5 mm thick and extends out past the labial rail 22 and the palatal rail 24 about 5.0 mm to about 10.0 mm. While the occlusal area 14 is discussed as having predetermined lengths on either side of the labial and palatal rails 22, 24, these dimensions can vary since they may be trimmed back to a variety of lengths by the user.

When inserted into the mouth, the rails 22, 24 act as retention members to help hold the mouthpiece system 1 in the user's mouth. The mouthpiece system 1 is inserted in the user's mouth so that the palatal rail 24 is placed behind the teeth. This rail 24 helps prevent the mouthpiece system 1 from sliding out of the mouth and improves the function of the mouthpiece system 1 by providing a more stable and fixed structure in the mouth.

As shown in FIG. 1, the palatal rail 24 can have a curved buttress 30 positioned [on each end of the occlusal area 14]. The curved buttress 30 allows the user's teeth to slide over the labial rail 22 for ease of insertion or removal. The labial rail 22 lies along the outside of and conforms to the occlusal area 14. The labial rail 22, among other things, can prevent the mouthpiece system 1 from sliding into the user's mouth.

A pad 26, such as a bumper or cushion can be attached to the bite area 6. The pad 26 can be situated along the occlusal area 14, between the labial rail 22 and the palatal rail 24, or covering a combination of any of the occlusal area 14 and the rails 22, 24. The pad 26 cushions the teeth and helps retain the mouthpiece within the user's mouth. The pad 26 is made of a cushioning material, such as an adhesive-backed medical foam, a pliable wax or plastic, or other non-toxic moldable material known by one skilled in the art to provide a cushioned surface.

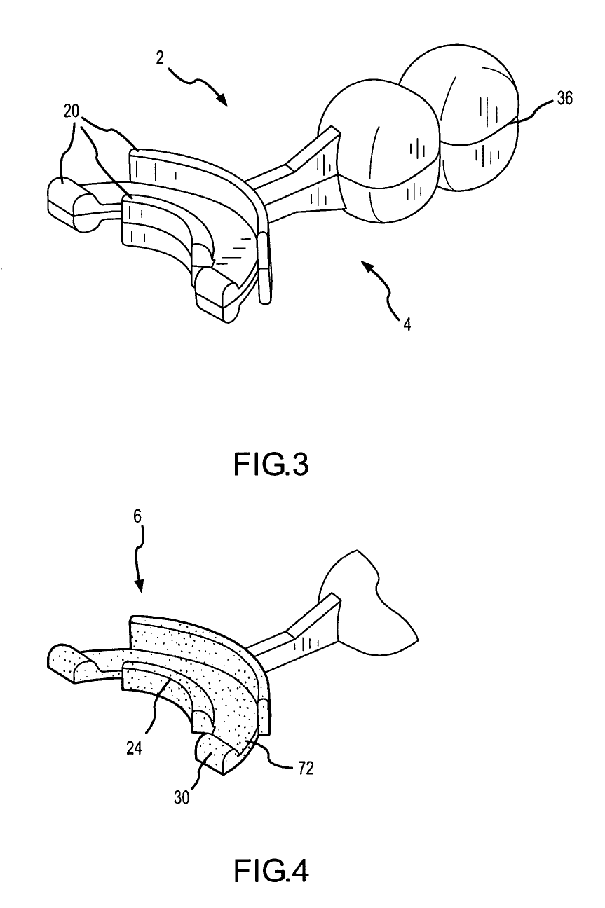

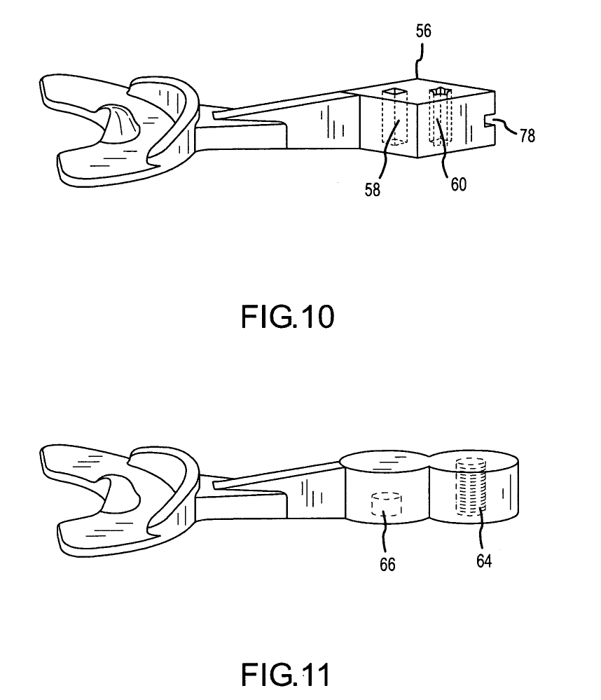

With respect to FIG. 2, the interface area 38 includes a raised body 40. The raised body 40 provides additional surface area and material to allow for a wide variety of alterations, connections, and configurations with medical, dental or other equipment. The raised body 40 includes a side surface 46 that can be rounded to form a cylindrical shape as shown in FIG. 2. In addition, the raised body 40 can include spherical bodies as discussed below with respect to FIG. 1 and shown in FIGS. 3-9 and 12-17, two cylinders as shown in FIG. 11, trapezoidal as shown in FIG. 10, and/or any other shape that would allow for alterations, connections, and configurations with peripheral equipment.

In the exemplary embodiment shown in FIG. 1, the interface area 38 includes two spherical bodies 48, 48′ that are interconnected and each having flat sides 42 on both sides of the spherical bodies 48, 48′. As discussed above, the bottom of the interface area 38 includes a flat surface making each of the spherical bodies 48, 48′ shaped like conjoined half-spheres with flat sides. One spherical body 48 is positioned inward from the other 48′. The inner spherical body 48 is attached to the extension bar 28 at a mid section of the sphere-like shape. The outer spherical body 48′ is interconnected to the inner spherical body 48. The mouthpiece system 1 is aligned to position the bite area 6, the extension bar 28 and the spherical bodies 48, 48′ along axis X as shown in FIG. 1.

The inner spherical body 48 and/or the outer spherical body 48′ include a set of pivots 44 on both flat sides 42. The spherical bodies 48, 48′ and flat sides 42 allow vertical and axial rotation of the mouthpiece system 1. The pivots 43 are rounded having a diameter of about 5.0 mm and a convex surface 44 shaped to align with the radius of the spherical bodies 48, 48′. The pivots 43 protrude from each of the flat sides 42 about 1.5 mm to about 3.0 mm to the crest of the convex surface 44. The pivots 43 can also have slots 45 in the sides of the radius and/or the convex surface to assist the user in aligning and adjusting other dental equipment to the mouthpiece system 1.

Each of the spherical bodies 48, 48′ can include a cavity 50 that is tunneled through each sphere-like shape perpendicular to axis X. The cavity 50 can have a variety of shapes. For example, cavities 50 shown in FIG. 1 have a vertical, cylindrical shape with a diameter of about 1.0 mm to about 8.0 mm. The cavity 50 can be a straight bore through the spherical bodies 48, 44, or a partial bore. The cavity 50 can also be threaded to allow a fastener to fit or fasten to the interface area 38. The two vertical cavities 50 illustrated in FIG. 1 are both straight, cylindrical bores.

The cavity 50 and/or the pivots 43 act as sockets for connecting other peripheral equipment. The cavity 50 is generally shaped to slide along a rod, or threaded for connecting to a fastener or other equipment, such as a threaded rod, bolt or screw. The pivot 43 can further act as a connection point for a rotating joint, like a ball and socket connection. The interface area 38 can also include a notch 78 formed or cut into the top side of the interface area 38. The notches 78 can be positioned toward the user's mouth, which could be used to help grip, stabilize, connect with or manipulate the interface area 38 and the overall mouthpiece system 1.

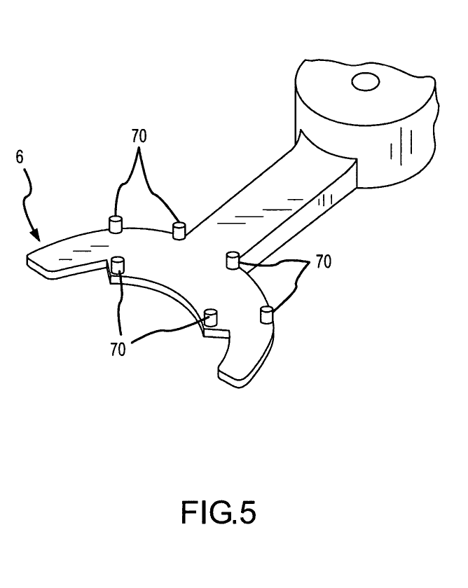

A resting surface 36 can extend along the bottom surface of the interface area 38, the extension bar 28, and the bite area 6 of the mouthpiece system 1. As shown in FIG. 3, the mouthpiece system 1 can include an upper mouthpiece portion 2 and a lower mouthpiece portion 4 placed together along the resting surface 36. The resting surface 36 is substantially flat to allow the upper mouthpiece portion 2 mate to the lower mouthpiece portion 4 when the lower mouthpiece portion is inverted. This way the mouthpiece system 1 can be used to fit the upper and lower teeth of a user. In addition, as shown in FIG. 4, the bite area 6 can include a buttress 30 positioned on each side of the occlusal area 14 at the molar edge 12. The buttress 30 is placed on the molar edge 12 to help align the mouthpiece system 1 with the user's teeth. Each buttress 30 is about 0.5 mm to 2 mm high and about 1 mm to 5 mm wide. In this embodiment, the buttress 30 has a curved upper surface with a radius of about 1.5 mm.

FIG. 17 shows another example of a mouthpiece system 1 having upper and lower mouthpiece portions 2, 4. In this exemplary embodiment, the two mouthpiece portions 2, 4 can be connected through a push-pull cable system 102 to a drive and control system 116. Other devices could further include a connecting system like a threaded rod, a ratcheting apparatus, or slide structure, which is connected through the push-pull cable system 102. The mouthpiece system 1 is connected to the push-pull cable system 102 through a conduit 110 at the interface area 38. The interface area 38 of the lower mouthpiece portion 4 includes a cavity 114 that has a cylindrical shape and is widened and threaded to allow a threaded conduit cap 112 to be inserted into the cavity 114.

The upper mouthpiece portion 2 includes a cavity 108 that is cylindrical shaped and threaded. The upper mouthpiece portion 2 is connected to a cable 104 through the cavity 108. The cable 104 is attached to a threaded cable cap 106 that is inserted into the cavity 108. In this mouthpiece system 1, the upper and lower mouthpiece portions 2, 4 are positioned inversely with their resting surfaces 36 facing each other and the occlusal area 14 facing outward.

The mouthpiece system 1 is inserted into the user's mouth by placing the bite area 6 of the upper mouthpiece portion 2 over the upper teeth and the bite area 6 of the lower mouthpiece portion 4 over the lower teeth. The user's teeth are placed between the labial rail 22 and the palatal rail 24. The drive and control system 116 uses the push-pull cable system 102 to move the cable 104 within the conduit 110. The cable 104 moves the upper mouthpiece portion 2 in relation to the lower mouthpiece portion 4 to increase or decrease the distance between the mouthpiece portions 2, 4, which opens or closes the user's mouth and jaw. This action provides motion to and/or stretching of the user's jaw, joint and mouth.

FIGS. 4 through 7 illustrate several of alternative embodiments of the mouthpiece system 1 having a bite area 6 with various arrangements. FIG. 4, for example, shows a coating 72 applied to the bite area 6. The coating 72 can substantially cover the entire surface of the bite area 6 to provide better grip and/or cushion for the user's teeth. The coating 72 can be made of a soft material such as an over-molded or dipped soft plastic, resin or foam.

FIG. 5 shows another embodiment of retention members 20 arranged in the bite area 6. In this embodiment, the retention members 20 include nubs 70 positioned along the bite area 6 to bracket the user's teeth over the occlusal area 14. The nubs 70 are about 1 mm in diameter and about 3 mm high. In this embodiment, the mouthpiece system 1 includes two nubs 70 spaced on the bite area 6 to align the mouthpiece system 1 against the back side of the user's teeth and four nubs 70 spaced along the bite area 6 to align the front side of the user's teeth.

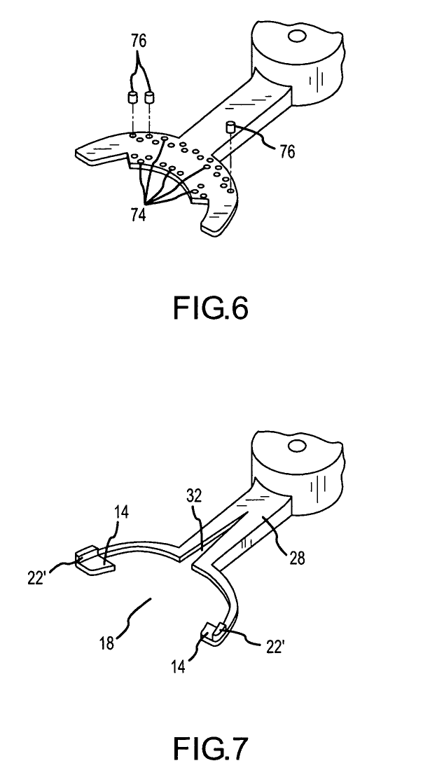

FIG. 6 shows a further embodiment of a mouthpiece system 1 including a bite area 6 with an arrangement of sockets 74 and a set of pins 76. In this embodiment, the sockets 74 are placed in the bite area 6 in a predetermined pattern, such as one or more rows, a grouping, one or more radial lines, circular pattern, or the like. The pattern allows the user to selectively place the set of pins 76 in the sockets 74 to customize the fit of the mouthpiece system 1. The set of pins 76 are made to press-fit into the sockets 74. The set of pins 76 can be placed at any point along the front and back of the user's teeth where the sockets 74 are located. More or less pins 76 can be used depending on whether the user needs more or less support to align the mouthpiece system 1.

FIG. 7 depicts a further embodiment of a mouthpiece system 1 including a bite area 6 with an extended oral cavity 18. The extended oral cavity 18 can be used in applications where the user requires more clearance around the user's teeth due to a large bit area and/or when the other dental equipment used with the mouthpiece system 1 requires additional clearance. The extension bar 28 in this embodiment also includes a split 32 in the center of the extension bar 28, which can add flexibility in the bite area 6 and allow the occlusal areas 14 to widen around opposing sides of the user's mouth. A modified labial rail 22′ rises up from the occlusal areas 14. These rails 22′ can act to squeeze against the users dentition to further hold the mouthpiece system 1 in place in the mouth. This embodiment of the mouthpiece system 1 also provides additional clearance around the occlusal area 14 to reduce contact with the user's front teeth.

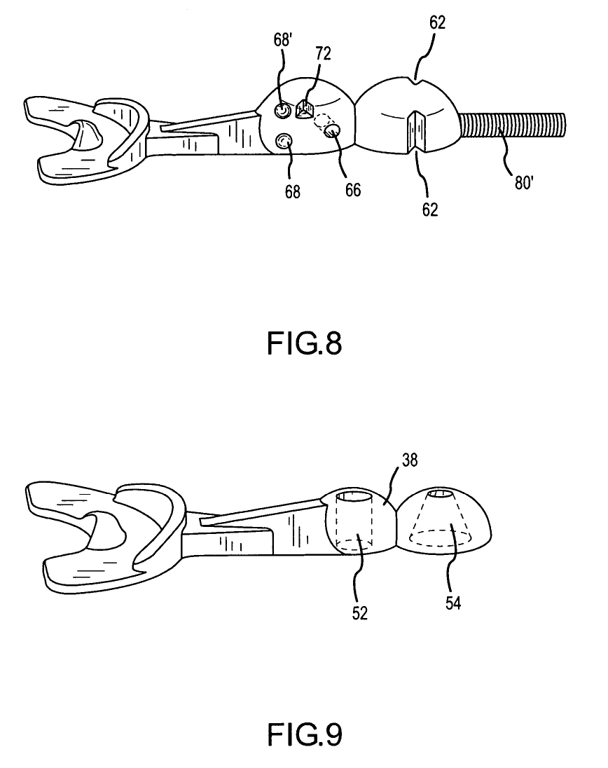

In various other embodiments of the invention, the interface area 38 is modified to accommodate other dental attachments, connections and equipment as shown in FIGS. 8-13. For example, FIG. 8 shows some alternative variations for modifying the surface and sides of the interface area 38. The interface area 38 can include a partial bore 66, a dimple inverted 68 and raised 68′, a notch 78 or a groove 62, which are carved, cut or formed into the interface area 38. The groove 62 is cut or formed in a side of the interface area 38 having a v-shape, c-channel shape, u-shape or other shape known by one skilled in the art to form or cut a groove. The groove 62 can be formed on one side and another one on the opposing side to allow an attachment to slide onto the interface area 38. A threaded extension 80′ extends out from the interface area as an additional modification to the interface area 38. These modifications can be added, subtracted and arranged to accommodate various attachments, covers, grips, secondary components or other interacting mechanisms to interconnect or work with the mouthpiece system 1.

In FIG. 9, a mouthpiece system 1 is illustrated having an interface area 38 that has a reduced thickness as compared to the embodiment shown in FIG. 8. Typically the thickness if the interface area 38 is between about 5 mm and about 50 mm. The thickness of the interface area 38 shown in FIG. 8 is about 18 mm and in FIG. 9 is about 10 mm. Although the thickness of the interface area 38 can vary depending on the external attachments and/or uses, the thicknesses described above are exemplary in nature. FIG. 9 also illustrates other variations of the cavity 50. For example, the cavity 50 can include a widened surface 52, where the diameter increased from the embodiment shown in FIG. 4. Typically, the diameter of the cavity is between about 1.0 mm and about 8.0 mm as discussed above. Here, however, the cavity has been increased to a diameter between about 8.0 mm to about 15 mm, where the diameter remains at least about 2.0 mm less than the width of the material of the interface area 38 to provide at least a 1.0 mm wall around the cavity 50. The interface area 38 can also include a cavity having a sloped surface 54. The sloped surface 54 can provide a conical-shaped bore, for instance. The sloped surface 54 can be determined by changing the opening of the cavity 50 from one end to the other. For example, the cavity 50 can have a diameter of about 2.0 mm on one end and about 9.0 mm on the other end. The openings of the cavity 50 can be inline or offset to create the sloped surface 54.

With respect to FIG. 10, the mouthpiece system 1 is illustrated having an interface area 38 with an alternative shape and arrangement. For example, the interface area 38 can have a parallelepiped shape 56. In addition, the interface area 38 can include a cavity 50 having a rectangular cross-section 58 or other geometric shape cross-section 60, such as a hexagon, octagon, x-shape, star-shape, u-shape, v-shape, or other geometric configuration. Also, a notch 78 is illustrated in FIG. 10 cut out or formed through the side of the interface area 38 from one side to another side substantially perpendicular to the top surface.

FIG. 11 depicts further embodiments of the mouthpiece system 1 having alternative arrangements of the cavity 50. The cavity 50 is shown as having threads 64, which is threaded to accommodate a screw, bolt, or other fastener to connect an attachment to the cavity 50. The cavity 50 can be drilled, bored, formed or otherwise cut through the interface area 38 from top to bottom or side to side. The cavity 50 can also be a partial bore 66 as shown in FIG. 11.

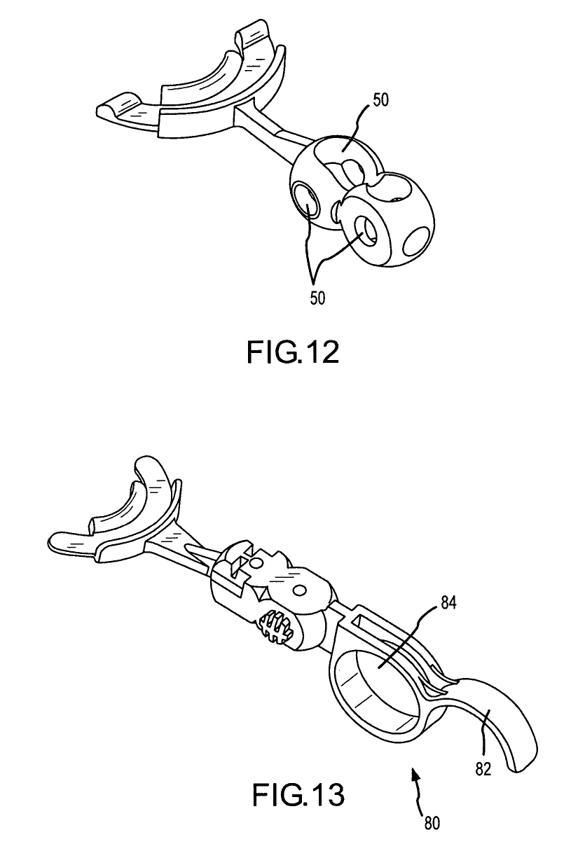

In FIG. 12, the mouthpiece system 1 is illustrated having the interface area 38 with the spherical body 48. The interface area 38 includes a cavity 50 arranged from side to side through the entire thickness. Another cavity 50 is drilled at an angle from the top down to the side to side cavity. In this manner, the cavity arrangement, as one example, allows the mouthpiece system 1 to be attached to a mechanism, such as a straight rod. The mouthpiece system 1 can then rotate and/or slide on the rod for a predetermined distance. Additional pivot components could be added to the angled cavity to assist in the rotation.

Another embodiment of the mouthpiece system 1 is shown in FIG. 13 with a contoured handle 80. The handle 80 is connected to the interface area 38 opposing the bite area 6. The handle 80 can be detachable or permanently affixed. For example, a removable fastener can attach the handle 80 to the interface area 38. This embodiment also illustrates the handle 80 having an open finger loop 82 and a closed finger loop 84 to allow a user to grip the mouthpiece system 1. The handle 80 can include other variations to allow a user to grip the mouthpiece system, for example, a cylindrical grip, a ball grip, or other various handle shapes known by one skilled in the art.

These figures show cavities most often in a vertical arrangement in the interface area 38 for clarity, however, the mouthpiece system 1 can include any number of cavities or protrusions in any direction. Furthermore, these variations only highlight the types of shapes and cavities possible, and can also be applied to other areas of the mouthpiece system as well.

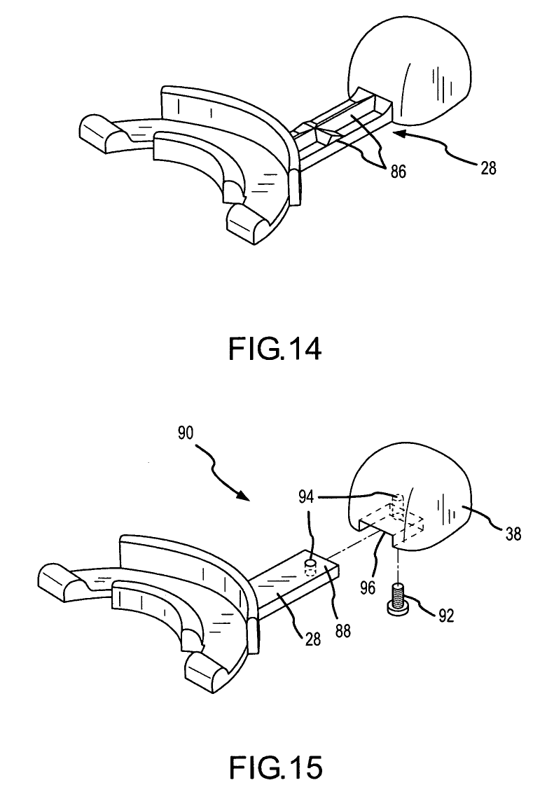

FIGS. 14-16 display possible variations of the extension bar 28 of the mouthpiece system 1. FIG. 14 shows the extension bar 28 with ridges 86 arranged along its span and width. These ridges 86 can provide extra support to the bar or act as measurement indicators. The extension bar 28 can also be made of a flexible material to allow a predetermined bend in the mouthpiece system 1 to respond to its movement and positioning.

Regarding FIG. 15, the mouthpiece system 1 includes a detachable clip structure 90 positioned between the interface area 38 and the bite area 6. The clip structure 90 in FIG. 15 includes an insert 88 and a receiver 96. The insert 88 is arranged on the extension bar 28 at the end abutting the interface area 38. The receiver 96 is cut or formed into the interface area 38. The extension bar 28 is assembled with the interface area 38 by placing the insert 88 into the receiver 96. A bore 94 can be placed in the insert 88 and the receiver 96. A fastener 92 can then be inserted into the bore 94 to secure the insert 88 to the receiver 96 when the bore 94 is aligned.

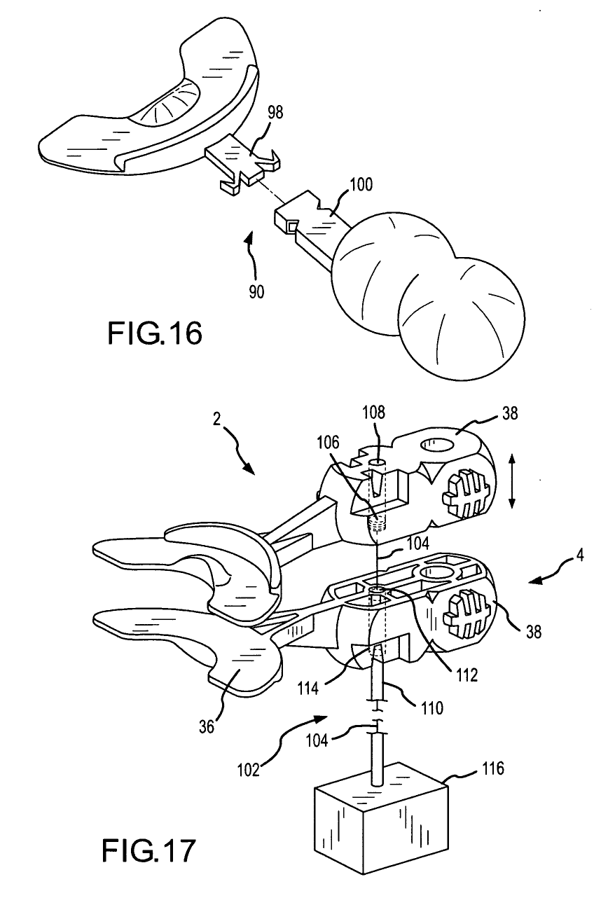

Another embodiment of the mouthpiece system 1 having a clip structure 90 is illustrated in FIG. 16. The clip structure 90 includes a clasp plug 98 attached to one portion of the extension bar 28 and a clasp socket 100 attached to another portion of the extension bar 28. The clasp plug 98 is made to insert into the clasp socket 100 and lock into place with a hooking mechanism. This embodiment of the mouthpiece system 1 allows a user to replace or interchange a bite area 6 or interface area 38 if, for example, it becomes broken, dirty, or requires switching out to be adapted to another component. This clip structure 90 can be further configured within or between any areas of the mouthpiece system 1 even though the illustrated embodiment shows it in the mid-portion of the extension bar 28. Additionally, detachable structures can be arranged on the mouthpiece system 1 as hinges to allow for a predetermined degree of movement within the mouthpiece system 1 to achieve various responses of manipulation and engagement of the user's mouth, face, and jaw.

Accordingly, it can be appreciated that that the present invention provides a mouthpiece system 1 that can be modified and configured for a variety of varying functions or to be used with a variety of devices. The mouthpiece system 1 provides a general shape to fit a variety of upper and lower bite patterns for users' teeth, as well as easy adaptation and connection to parts of independent devices. The design of the mouthpiece system 1 allows for economical manufacturing and a robust structure to include multiple interface structures or to receive various secondary alterations. Furthermore, the mouthpiece system 1 has many additional advantages, which can include:
a) requiring no custom manufacturing by incorporating a variety of arrangements including a wide occlusal area 14 and retention members 20 with varying heights to fit a variety of mouths, and/or provided with a thickness that is easily cut or trimmed;
b) being interchangeable with exterior components;
c) having a structure small enough to fit between the teeth of users with severe restrictions in their opening, while holding the mouthpiece system 1 within the mouth between the teeth;
d) providing an area to hold a padded or moldable material for improved comfort and retention; and
e) having an interface or control area for the interaction, connection and manipulation of the mouthpiece system 1 to engage the mouth and jaw of a user.

While the above description contains many specific examples, these exemplary embodiments should not be construed as limitations on the scope of the invention but as exemplifications of the present embodiments. Many other ramifications and variations are possible within the teachings of the present invention. For example, the different areas of the mouthpiece system 1 can have different shapes, sizes and cavities; new unspecified additions to the biteplate, bar or interface area can be added; any non-toxic stiff or semi-stiff materials in any color can be used; any non-toxic pad, cushion, and adhesive can be used; a single mouthpiece can be made to engage both upper and lower teeth; and the invention can be cored out specifically to match its features for manufacturing and injection molding.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A mouthpiece system comprising:
a. a bite area shaped to fit a general curved pattern of a user's dentition, the bite area including an occlusal area arranged to contact the user's dentition when inserted into the user's mouth and having a palatal edge on a side proximate the user's throat and a labial edge on another side proximate the user's lips, and a molar edge proximate the user's molar teeth;
b. an interface area including a raised body; and
c. an extension bar connected between the bite area and the interface area.

2. The mouthpiece system of claim 1, wherein the bite area, the interface area, and the extension bar are formed as a continuous unit.

3. The mouthpiece system of claim 1, further comprising at least one retention member placed on the occlusal area, the retention member being a raised wall to be located near at least one of the molar, palatal and labial edges of the occlusal area.

4. The mouthpiece system of claim 1, further comprising a pad placed along the occlusal area.

5. The mouthpiece system of claim 1, further comprising at least one cavity arranged on the interface area.

6. The mouthpiece system of claim 5, wherein the cavity includes at least one of a bore, a groove, a partial bore, a notch, a nub, a pivot, and a dimple.

7. The mouthpiece system of claim 1, further comprising a plurality of sockets formed in the occlusal area and pins to be inserted into the sockets.

8. The mouthpiece system of claim 1, further comprising a clip structure positioned between the interface area and the bite area and arranged to detachably connect the interface area with the bite area.

9. The mouthpiece system of claim 8, wherein the clip structure includes an insert and receiver, the insert being configured to fit into the receiver to connect the bite area with the interface area.

10. The mouthpiece system of claim 8, wherein the clip structure includes a clasp plug and a clasp socket, the clasp plug being configured to fit into the clasp socket to connect the bite area with the interface area.

11. The mouthpiece system of claim 1, further comprising an upper mouthpiece portion and a lower mouthpiece portion, each of the upper and lower mouthpiece portions including the bite area, the interface area, and the extension bar.

12. The mouthpiece system of claim 11, wherein each of the upper and lower mouthpiece portions include a resting surface, the resting surface of the upper mouthpiece portion being configured to match the resting surface of the lower mouthpiece portion when the upper and lower mouthpiece portions are placed in opposing directions to fit an upper and lower set of teeth.

13. The mouthpiece system of claim 1, further comprising a rail placed on at least one of the labial and palatal edge.

14. The mouthpiece system of claim 11, further comprising a push-pull cable of a conduit, a conduit cap, a cable, and a cable cap, wherein the cable runs through the conduit, the cable cap attaches to one end of the cable, the conduit cap attaches to corresponding end of the conduit, the cable cap attaches to the upper mouthpiece portion and the conduit cap attaches to the lower mouthpiece portion.

15. The mouthpiece system of claim 1, further comprising at least one buttress placed on the molar edge.

16. The mouthpiece system of claim 1, further comprising a split formed in the extension bar.

17. The mouthpiece system of claim 1, further comprising a flared edge attached to the extension bar between the bite area and the interface area.

18. The mouthpiece system of claim 1, wherein the raised body includes at least one spherical body.

19. The mouthpiece system of claim 1, further comprising a coating applied to the bite area.

20. The mouthpiece system of claim 1, further comprising a handle attached to the interface area.

21. The mouthpiece system of claim 1, further comprising at least one ridge attached to the extension bar.

22. A method of making a mouthpiece system, the method comprising:
a. forming a bite area shaped to fit a general curved pattern of a user's dentition, the bite area including an occlusal area arranged to contact at least a portion of the user's dentition when inserted into the user's mouth and having a palatal edge on a side proximate the user's throat and a labial edge on another side proximate the user's lips, and a molar edge proximate the user's molar teeth;
b. forming an interface area including a raised body; and
c. forming an extension bar to be connected between the bite area and the interface area.

23. The method of claim 22, further comprising a step of forming at least one retention member on the occlusal area.

24. The method of claim 22, further comprising a step of attaching a pad along the occlusal area.

25. The method of claim 22, further comprising a step of forming at least one cavity in the interface area.

26. The method of claim 22, further comprising a step of forming a plurality of sockets in the occlusal area, and inserting pins into the sockets.

27. The method of claim 22, further comprising a step of forming another mouthpiece, and integrating a cable with a cable cap, and a conduit with a conduit cap in a push-pull cable system, and connecting the push-pull cable system with the cable connected to one of the mouthpieces and the conduit connected to the other one of the mouthpieces.

28. The method of claim 22, further comprising a step of forming a split in the extension bar.

29. The method of claim 22, further comprising a step of applying a coating to the bite area.

30. The method of claim 22, further comprising a step of connecting the extension bar between the bite area and the interface area with a clip structure.
